(19) United States
(12) Patent Application Publication
(10) Pub. No.: US 2019/0175326 A1
(43) Pub. Date: Jun. 13, 2019

(54) Neurovascular Stent

(71) Applicant: MICO Innovations, LLC, Rockville Centre, NY (US)

(72) Inventors: Steven Lisi, Rockville Centre, NY (US); Carl Belczynski, Rockville Centre, NY (US)

(21) Appl. No.: 16/327,486

(22) PCT Filed: Aug. 24, 2017
(86) PCT No.: PCT/US2017/048403

§ 371 Date: Feb. 22, 2019

Related U.S. Application Data
(60) Provisional application No. 62/379,467, filed on Aug. 25, 2016.

(51) Int. Cl. A61F 2/07; A61F 2/91; A61F 2/92

(52) U.S. Cl. CPC A61F 2/07; A61F 2/91; A61L 31/14; A61F 2250/0041; A61F 2/92

(57) ABSTRACT

An intravascular stent wherein at least a portion of the stent is formed of a refractory alloy. At least 95 wt. % of the refractory alloy is formed of two refractory metals. The refractory alloy is a non-shape memory alloy and a non-self-expanding alloy. The stent has a wall thickness, strut thickness and strut configuration to enable it to be expanded from an unexpanded configuration to a fully expanded configuration by expansion pressure of less than about 6 atm. by an inflatable device inflating against an inside surface of said metal tube body. The stent has sufficient radial strength in the fully expanded position to resist deformation when exposed to an external radial pressure of over 1 atm.

The present invention claims priority on U.S. Provisional Application Ser. No. 62/379,467 filed Aug. 25, 2016, which is incorporated herein by reference.

The present invention relates generally to the treatment of neurological conditions, particularly to intravascular medical devices formed of a refractory non-memory alloy, where at least 95 wt. % of the refractory non-memory alloy is formed of two refractory metals, and which is non-self-expanding and is expandable at low pressures. More particularly, the present invention relates to an expandable stent/graph formed of a non-memory alloy that includes a majority of tantalum and tungsten, and which is non-self-expanding and is expandable at low pressures. The invention is also directed to a novel treatment method that delivers the medical device formed of a non-memory alloy that includes a majority of tantalum and tungsten to a complex vessel at ultra-low pressure (less than 6 atm.), wherein the non-memory alloy of the medical device has intrinsic properties which enable the medical device to be expandable at lower pressure without cracking or causing other damage to the medical device during the expansion of the medical device, and which medical device is non-self-expanding. Such devices are useful in treating blood vessels in the brain.

### BACKGROUND OF THE INVENTION

Medical treatment of various illnesses or diseases commonly includes the use of one or more medical devices. Two types of medical devices that are commonly used to repair various types of body passageways are an expandable graft or stent, or a surgical graft. These devices have been implanted in various areas of the mammalian anatomy. One purpose of a stent is to open a blocked or partially blocked body passageway. In a blood vessel, the stent is used to open the occluded vessel to achieve improved blood flow, which is necessary to provide for the anatomical function of an organ. The procedure of opening a blocked or partially blocked body passageway commonly includes the use of one or more stents in combination with other medical devices such as, but not limited to, an introducer sheath, a guiding catheter, a guide wire and an angioplasty balloon, and pharmaceutical or chemical delivery agents, etc.

In a balloon-expanding stent, an inflatable device, such as a “balloon”, is inflated to expand the stent to the vessel diameter at the delivery site. The balloon is then deflated and removed to leave the stent in place. An alternative to the balloon-expanding stent is a self-expanding stent (manufactured at the vessel diameter) which deploys at the delivery site when a constraint is removed. Self-expanding stents display elastic properties and assist in balloon inflation. Balloon-expanding stents, however, resist the balloon. Therefore, balloon-expanding stents require greater expansion pressures than self-expanding stents to deploy during the stenting procedure. The materials that are selected to form a medical device and, more particularly, a balloon-expanding verses a self-expanding medical device, are based on their expansion properties and material composition.

Various other physical attributes of a stent and balloon catheter delivery system can contribute directly to the success rate of the device. These physical attributes include radiopacity, hoop strength, radial strength/force, radial stiffness, radial compliance, acute recoil, thickness of the metal, dimensions of the metal and the like. Cobalt chromium-, platinum chromium-, and nitinol-based alloys are commonly used to form stents and display physical characteristics that allow for certain design and functional features, such as a thin strut pattern. Nitinol is a well-known shape memory alloy that displays a superelastic characteristic when exposed to body temperature, meaning that it can adapt to the shape of the vessel. Nitinol- and platinum-based alloys are commonly used in struts for their self-expanding properties. These materials have been commonly used to form prior self-expanding stents since such materials have a known history of safety, effectiveness, ease of manufacturing and biocompatibility.

Alternative materials, such as stainless steel, are common to balloon-expanding stents. Although these materials are expandable under pressure using an inflatable device, they require certain expansion pressures to deploy. As such, the risks associated with stent implantation (such as artery puncture and over-expansion at high pressures) can cause damage to the lining of the vessel (causing an artery dissection), and inflammatory response can be related to the pressure used to deploy the stent in the target vessel. While coronary stents have demonstrated great success during high- and low-pressure placement in cardiovascular vessels, a similar low-pressure deployment (e.g., 8-13 atmospheres (“atm.”)) in the vessels in the brain can add risk and complication during the deployment of the stent, causing physician concerns and can result in damage or rupture of the vessel in the brain.

Particularly, the vessels inside the brain are smaller, more complex and more fragile than coronary arteries, thus require low pressure on the vessel walls. As further compared to other vessels, the difficult-to-reach twists and turns of intracranial vessels require a brain stent be manufactured from a more flexible stent and delivery system. Brain stents are devices implanted in the brain to help patients who suffer strokes caused by clots that block blood flow. Brain stents can also be used to treat intracranial atherosclerosis disease (ICAD)—sometimes called “hardening of the arteries”. ICAD occurs when these arteries become clogged with plaque thereby limiting blood flow to the brain and increasing the risk of a stroke. Brain stents are also used to treat other high risk neurological conditions, such as brain aneurysm, intracranial stenosis, and thrombosis, etc. Certain pharmacologic treatments using stents must begin within hours of the onset of symptoms. Upon deployment, however, the stent can clear the clot, maintain blood flow in narrowing vessels, and treat aneurysms to lower the patient's risk of brain damage or death.

Therefore, a medical treatment is desired for treating neurological conditions, such as stroke, aneurysm, ICAD, and stenosis, among others, and which is assisted by delivery of a medical device manufactured from a material that is expandable under lower pressures for neurological applications, especially when pharmacologic treatments are contraindicated due to patient presentation outside the window of opportunity.

### SUMMARY OF THE INVENTION

The current invention is generally directed to a method for treatment of neurological conditions using a medical device that is at least partially formed of a refractory alloy that includes two primary metals, namely tantalum and tungsten, the amount of tantalum in the refractory alloy is greater than the amount of tungsten in the refractory alloy, and which medical device is non-self-expanding and is expandable at low pressures. The medical device can also incorporate one or more specific design features and/or surface modifications that enhance one or more of the physical properties of the medical device so as to improve the success rate of such medical device in the treatment of certain neurological conditions and to overcome several of the past problems associated with such medical devices.

One non-limiting embodiment of the disclosure is directed to an intravascular stent comprising a strut pattern cut (e.g., laser cut, etc.) from a non-clad metal tube body. At least a portion of the non-clad metal tube body is formed of a refractory alloy that is non-self-expanding alloy and which body that is formed from the non-self-expanding alloy is expandable at low pressures. In one specific embodiment, about 80-100% of the non-clad metal tube body (and all values and ranges therebetween) is formed of the refractory alloy that is non-self-expanding alloy and which body that is formed from the non-self-expanding alloy is expandable at low pressures. At least 95 wt. % of the refractory alloy is formed of two refractory metals. In one non-limiting embodiment, about 98-100 wt. % of the refractory alloy is formed of two refractory metals, namely tantalum and tungsten. The metal tube is a non-shape memory alloy and a non-self-expanding alloy. The stent is partially or fully formed of a refractory alloy that is a non-shape memory alloy and a non-self-expanding alloy and has a wall thickness, strut thickness and strut configuration to enable it to be expanded from an unexpanded configuration to a fully expanded configuration by an expansion pressure of greater than 1 atm. and requiring less than 6 atm. (and all values and ranges therebetween) to be fully expanded by an inflatable device inflating against an inside surface of said metal tube body. In one non-limiting embodiment, the stent is partially or fully formed of a refractory alloy that is a non-shape memory alloy and a non-self-expanding alloy that is expandable to a fully expanded configuration by an expansion pressure of greater than 1 atm. and requiring no more than 5 atm. to be fully expanded by an inflatable device inflating against an inside surface of said metal tube body. In another non-limiting embodiment, the stent is partially or fully formed of a refractory alloy that is a non-shape memory alloy and a non-self-expanding alloy that is expandable to a fully expanded configuration by an expansion pressure of greater than 1 atm. and requiring no more than 4 atm. to be fully expanded by an inflatable device inflating against an inside surface of said metal tube body. In another non-limiting embodiment, the stent is partially or fully formed of a refractory alloy that is a non-shape memory alloy and a non-self-expanding alloy that is expandable to a fully expanded configuration by an expansion pressure of greater than 1 atm. and requiring no more than 3 atm. to be fully expanded by an inflatable device inflating against an inside surface of said metal tube body. In another non-limiting embodiment, the stent is partially or fully formed of a refractory alloy that is a non-shape memory alloy and a non-self-expanding alloy that is expandable to a fully expanded configuration by an expansion pressure of greater than 1 atm. and requiring no more than 2 atm. to be fully expanded by an inflatable device inflating against an inside surface of said metal tube body. The metal tube body in the unexpanded configuration is configured to enable the stent to be inserted into a body passageway. The stent in the fully expanded positioned is configured to engage an inner wall of the body passageway to support an opening in the body passageway. The stent has sufficient radial strength in the fully expanded position to resist deformation when exposed to an external radial pressure of greater than 1 atm. In one non-limiting embodiment, the stent has sufficient radial strength in the fully expanded position to resist deformation when exposed to an external radial pressure of greater than 1 atm. and less than 6 atm. (and all values and ranges therebetween). In another non-limiting embodiment, the stent has sufficient radial strength in the fully expanded position to resist deformation when exposed to an external radial pressure of greater than 1 atm. and up to 5 atm. In another non-limiting embodiment, the stent has sufficient radial strength in the fully expanded position to resist deformation when exposed to an external radial pressure of greater than 1 atm. and up to 4 atm. In another non-limiting embodiment, the stent has sufficient radial strength in the fully expanded position to resist deformation when exposed to an external radial pressure of greater than 1 atm. and up to 3 atm. In another non-limiting embodiment, the stent has sufficient radial strength in the fully expanded position to resist deformation when exposed to an external radial pressure of greater than 1 atm. and up to 2 atm.

In another non-limiting embodiment of the invention is directed to an intravascular stent comprising a strut pattern cut from a non-clad metal tube body wherein the diameter of the stent in the fully unexpanded position is about 1 mm to less than about 5 mm in diameter (and all values and ranges therebetween), and the stent in the fully expanded state is about 2-12 mm in diameter (and all values and ranges therebetween). In another non-limiting embodiment, the diameter of the stent in the fully unexpanded position is about is about 1-4 mm in diameter, and the stent in said fully expanded state is about 2-12 mm in diameter. In another non-limiting embodiment, the diameter of the stent in the fully unexpanded position is about is about 1-3 mm in diameter, and the stent in said fully expanded state is about 2-10 mm in diameter. In another non-limiting embodiment, the diameter of the stent in the fully unexpanded position is about is about 1-2.5 mm in diameter, and the stent in said fully expanded state is about 2-8 mm in diameter. Traditional materials used to manufacture stents such as stainless steel, cobalt-chromium alloy and cobalt-nickel alloy cannot be used to successfully form a stent having unexpanded diameters of less than 5 mm. Stainless steel stents have not be successfully formed to have an unexpanded diameters of less than 10 mm and still be successfully expanded in a body passageway without damage to the stent structure. Recently used alloys such as cobalt-chromium alloy and cobalt-nickel alloy can be used to form stents having a smaller unexpanded diameter (e.g., about 7-10 mm) than a stainless steel stent, and still be expanded without damage to the stent structure. However, even these types of stents cannot be successfully formed to have a diameter in the fully unexpanded position that is less than about 7 mm, nor can such stents be fully expanded by balloon expansion using a pressure of less than about 6 atm. Such stents formed by these prior art alloys also exhibit cracking in the alloy structure during the cutting and/or processing of the alloy when forming the final stent having a diameter in the fully unexpanded position that is less than about 7 mm, thereby resulting in the failure (e.g., breaking or partial tearing of one or more struts, etc.) of the stent when expanded to the fully expanded position. The damage to the one or more struts of the expanded stent can result in the piercing or tearing of the inner surface of the body passageway when the stent is expanded, and/or compromising the structural integrity of the stent when in the expanded position thereby increasing the incidence that the stent can be caused to collapse in the body passageway. Also, such traditional materials used to manufacture stents typically require balloon pressures of greater than 6 atms. to cause the stent to fully expand from the unexpanded position. Due to the recoil properties of the traditional materials used to manufacture stents (e.g., recoil of stainless steel is about 8-10%), the stents were typically required to be over-expanded by at least 6% and typically about 7-10% so that when the traditional materials recoiled after being expanded, the expanded outer diameter of the stent maintained its positioned on the inner wall of the body passageway. For example, a stainless steel stent that was to be expanded to a final outer diameter of 10 mm, typically required the stent to be over-expanded to at least 11 mm in diameter to account for the 7-10% recoil that occurs after the expansion balloon is deflated. The stent formed of the novel refractory alloy of the present invention (e.g., at least 95 wt. % of alloy formed of tantalum and tungsten) overcomes these significant limitations associated with prior art stents, and such stent 1) can be successfully formed to have a diameter in the fully unexpanded position of no more than about 5 mm in diameter, 2) can be successfully expanded in a body passageway to the fully expanded position without damage or cracking of the struts of the stent, 3) after being expanded to the fully expanded position has a recoil of no more than 5%, typically more than about 4%, and more typically no more than about 3%, 4) can be fully expanded by balloon expansion using a pressure of less than about 6 atm., and 5) can be expanded by balloon expansion such that the stent is fully expanded without having to be over expanded by more than 5% (e.g., 0-5% and all values and ranges therebetween). Such a stent has significant advantages over prior art stents in that the stent of the present invention 1) can be formed into a smaller expanded shape so that the stent can be placed in smaller blood vessels in the brain as compared to larger stents formed from traditional material, 2) can be fully expanded at lower pressures (e.g., no more than 6 atm.) as compared to stents formed from traditional material so as to avoid damage to the more fragile blood vessel in the brain during the expansion of the stent, and 3) does not required to be over-expanded by more than 5% to maintain the desired final expanded diameter of the stent when fully expanded; thus, damage to the more fragile blood vessels in the brain during the expansion of the stent is reduced. Over-expanding a stent to more than 5% of the inner diameter of a brain blood vessel significantly increases the chance of damaging or rupturing the blood vessel.

In another non-limiting embodiment of the invention is directed to an intravascular stent comprising a strut pattern cut from a non-clad metal tube body wherein the strut thickness (e.g., as defined initially by the wall thickness of the tube from which the stent is cut) is no more than about 0.0029 inches when the stent is used for treating blood vessels in the brain. In one non-limiting embodiment, the strut thickness is about 0.001-0.0029 inches (and all values and ranges therebetween) when the stent is used for treating blood vessels in the brain. In another non-limiting embodiment, the strut thickness is about 0.0012-0.0022 inches when the stent is used for treating blood vessels in the brain. In another non-limiting embodiment, the strut thickness is about 0.0012-0.002 inches when the stent is used for treating blood vessels in the brain. In another non-limiting embodiment, the strut thickness is about 0.0012-0.0018 inches when the stent is used for treating blood vessels in the brain. In another non-limiting embodiment, the strut width is no more than about 0.0029 inches when the stent is used for treating blood vessels in the brain. In another non-limiting embodiment, the strut width is about 0.0002-0.0029 inches (and all values and ranges therebetween) when the stent is used for treating blood vessels in the brain. In another non-limiting embodiment, the strut width is about 0.0004-0.0022 inches when the stent is used for treating blood vessels in the brain. In another non-limiting embodiment, the strut width is about 0.0004-0.0020 inches when the stent is used for treating blood vessels in the brain. In another non-limiting embodiment, the strut width is about 0.0004-0.0018 inches when the stent is used for treating blood vessels in the brain. Generally, the ratio of the thickness of the strut to the width of the strut is 5:1 to 1:1 (and all values and ranges therebetween) when the stent is used for treating blood vessels in the brain. In another non-limiting embodiment, the ratio of the thickness of the strut to the width of the strut is 4.5:1 to 1.01:1 when the stent is used for treating blood vessels in the brain. In another non-limiting embodiment, the ratio of the thickness of the strut to the width of the strut is 4:1 to 1.01:1 when the stent is used for treating blood vessels in the brain. The small thicknesses and widths of the struts of the stent of the present invention are less than the thicknesses and widths of struts of stents formed by traditional materials. For example, when stents are formed from stainless steel, the thickness of the strut is generally at least 0.0045 inches. Use of a strut thickness of less than 0.0045 inches for a stainless steel stent will result in the cracking and/or breakage of the strut during the expansion of the stent. It is believed that if the stent in accordance with the present invention has a strut thickness of less than 0.001 inches, the recoil of the strut will increase to greater than 5%. The thickness of the strut on the stent in accordance with the present invention is generally constant; however, this is not required. The width of the strut on the stent in accordance with the present invention generally varies along the longitudinal length of the strut; however, this is not required.

Another non-limiting embodiment of the invention is directed to a method for treating a disease in a body passageway using an intravascular stent. The method includes the step of positioning the stent in a diseased vessel of the brain while it is in the unexpanded configuration. The method includes the step of expanding the stent that is partially or fully formed of a refractory alloy that is a non-shape memory alloy and a non-self-expanding alloy at a low pressure of greater than 1 atm. and less than 6 atm. from the unexpanded configuration to a fully expanded configuration using an inflatable device positioned in an interior of the stent. The stent in the fully expanded position has an outer surface engaging an inner surface of the diseased vessel and thereby causes it to be secured in a static positioned in the diseased vessel. The method further includes the step of deflating the inflatable device and removing the deflated inflatable device from the stent. The stent is further configured to have sufficient radial strength in the fully expanded position to resist deformation when exposed to an external radial pressure of greater than 1 atm. by the diseased vessel. During the expansion of the stent, the structure and composition of the stent is such that little or no over-expansion of the stent is required to fully expand the stent in the blood vessel. The blood vessels in the brain are more fragile and can be more easily damaged as compared to cardiac vessels. Typically, when a stent is expanded, it is over-expanded to ensure that the stent is fully expanded in the blood vessel. Prior art balloon expanded stents are made of materials that require the expansion balloon to be expanded at pressures typically exceeding 6 atm. for the balloon to properly expand the stent. During the expansion process, the stent is typically over-expanded by at least about 7% of the final diameter of the stent. For example, a cardiac stent that is to have a final expanded diameter of 3 mm is typically over-expanded to about 3.25 mm at an expansion pressure exceeding 6 atm. (e.g., about 8.3% overexpansion). Such high expansion pressures and degree of over expansion of the stent can be safely used for cardiac vessels due to the durability of the cardiac vessel. However, for blood vessels in the brain, such high expansion pressures and degree of over-expansion have a tendency to damage and/or rupture the blood vessel when the stent is expanded in the blood vessel. The present invention overcomes this significant limitation of prior art stents by provide a stent formed of a novel alloy and structure that enables the stent to be fully expanded by a balloon at pressures that are less than 6 atm. and typically less than 4 atm. and which the percentage of over-expansion of the stent as compared to the final fully expanded diameter is about 0-5% (and all values and ranges therebetween), typically about 0-4%, and more typically about 0-3%. For example, the stent of the present invention that is to have a final expanded diameter of 3 mm is typically over-expanded to no more than about 3.08 mm at an expansion pressure of less than 6 atm. (e.g., no more than 2.67% over-expansion). The combination of low expansion pressure and significantly less over-expansion during the expansion of the stent results in reduced incidence of damage to the blood vessel, especially the more fragile blood vessels in the brain. Such an advancement in the stent configuration and method of stent expansion represents an improvement in stent technology not before achieved.

In summary, there is provided an intravascular stent that has a strut pattern that is generally laser-cut from a non-clad metal tube body. As can be appreciated, the stent could be formed by 3-D printing. At least a portion of the non-clad metal tube body is formed of a refractory alloy. At least 95 wt. % of the refractory alloy is formed of two refractory metals. The metal tube is a non-shape memory alloy and a non-self-expanding alloy. The stent has a wall thickness, a strut thickness and a strut configuration to enable the stent to be expanded from an unexpanded configuration to a fully expanded configuration by expansion pressure of less than 6 atmosphere (atm.) from an inflatable device inflating against an inside surface of the metal tube body. The metal tube body in the unexpanded configuration is configured to enable the stent to be inserted into a body passageway. The stent in the fully expanded positioned is configured to engage an inner wall of the body passageway to support an opening in the body passageway. The stent is configured to have sufficient radial strength in the fully expanded position to resist deformation when exposed to an external radial pressure of over 1 atm.

In one non-limiting aspect of the invention, 98-100 wt. % of the refractory alloy used in the stent can be formed of two refractory metals. Generally, the total of amount of impurities and other metals in the stent that is formed of two refractory metals is about 0-2 wt. % (and all values and ranges therebetween), typically 0-1 wt. %, more typically 0-0.5 wt. %, still more typically 0-0.1 wt. %, ever more typically 0-0.01 wt. %, and still even more typically 0-0.005 wt. %.

In another or alternative non-limiting aspect of the invention, the two refractory metals in the refractory alloy are tantalum and tungsten.

In another or alternative non-limiting aspect of the invention, the refractory alloy consists essentially of 90-97.5 wt. % tantalum and a balance weight percent of tungsten.

In another or alternative non-limiting aspect of the invention, the stent is fully expandable from an unexpanded configuration to a fully expanded configuration by an expansion pressure of over 1 atm. and requires less than 6 atm. (and all values and ranges therebetween) for full expansion.

In another or alternative non-limiting aspect of the invention, the stent is fully expandable from an unexpanded configuration to a fully expanded configuration by an expansion pressure of over 1 atm. and requires no more than 5 atm. for full expansion.

In another or alternative non-limiting aspect of the invention, the stent is fully expandable from an unexpanded configuration to a fully expanded configuration by an expansion pressure of over 1 atm. and requires no more than 4 atm. for full expansion.

In another or alternative non-limiting aspect of the invention, the stent is fully expandable from an unexpanded configuration to a fully expanded configuration by said expansion pressure of over 1 atm. and requires no more than 3 atm. for full expansion.

In another or alternative non-limiting aspect of the invention, an exterior surface of the metal tube body of the stent includes surface modifications operative to receive a bioactive agent.

In another or alternative non-limiting aspect of the invention, there is provided a method for treating a disease in a body passageway using an intravascular stent comprising the steps of: 1) positioning the stent in a diseased vessel of the brain while the stent is in the unexpanded configuration; 2) expanding said stent at a low pressure of over 1 atm. and less than 6 atm. from the unexpanded configuration to the fully expanded configuration using an inflatable device positioned in an interior of the stent, and wherein the stent in the fully expanded position has an outer surface of the stent engaging an inner surface of the diseased vessel and thereby causing the stent to be secured in a static positioned in the diseased vessel; and 3) deflating the inflatable device and removing the deflated inflatable device from the stent; and wherein the stent is configured to have sufficient radial strength in the fully expanded position to resist deformation when exposed to an external radial pressure of over 1 atm. by the diseased vessel.

In another or alternative non-limiting aspect of the invention, the inflatable device is a balloon or inflatable catheter.

In another or alternative non-limiting aspect of the invention, the disease in the body passageway is a neurovascular disease that consists of intracranial artery stenosis, intracranial aneurysm, thrombus, or a combination of the above.

In another or alternative non-limiting aspect of the invention, the stent in the unexpanded configuration is about is about 1 to less than about 5 mm in diameter (and all values and ranges therebetween), and the stent in the stent in said fully expanded state is about 2-12 mm in diameter (and all values and ranges therebetween).

In another or alternative non-limiting aspect of the invention, the stent in the unexpanded configuration is about is about 1-4 mm in diameter, and the stent in the stent in said fully expanded state is about 2-12 mm in diameter.

In another or alternative non-limiting aspect of the invention, the stent in the unexpanded configuration is about is about 1-3 mm in diameter, and the stent in the stent in said fully expanded state is about 2-10 mm in diameter.

In another or alternative non-limiting aspect of the invention, the stent in the unexpanded configuration is about is about 1-2.5 mm in diameter, and the stent in the stent in said fully expanded state is about 2-8 mm in diameter.

In another or alternative non-limiting aspect of the invention, the stent in the unexpanded configuration is about 1-1.5 mm in diameter (and all values and ranges therebetween), and the stent in the stent in said fully expanded state is about 2-7 mm in diameter.

In another or alternative non-limiting aspect of the invention, the stent is over-expanded in the body passageway by 0-5% of the final fully expanded diameter of the stent in the body passageway using an inflatable device that is pressurize to less than 6 atm.

One non-limiting object of the present invention is the treatment of neurological conditions by stenting with a medical device that is formed of a metal alloy that includes tantalum and tungsten, wherein stenting with the tantalum-tungsten alloy-based medical device demonstrates improved procedural success rates.

Another and/or additional non-limiting object of the present invention is the provision of a medical device that can be used to treat of neurological conditions with a Ta—W alloy-based medical device wherein such device is expandable under substantially lower pressures than prior stents formed of other alloy materials.

Still another and/or additional non-limiting object of the present invention is the provision of a medical device that can be used to treat neurological conditions by stenting with a Ta—W alloy-based medical device using less than 6 atm. pressure to reduce risk of artery puncture, damage to the lining of the vessel (causing an artery dissection), and inflammatory response as compared to prior stenting using other medical devices.

Another and/or additional non-limiting object of the present invention is the provision of a medical device that reduces risks associated with the treatment of a blood vessel in the brain.

Another and/or additional non-limiting object of the present invention is the provision of a medical device that allows access to the smaller and more complex vessels of the brain.

Another and/or additional non-limiting object of the present invention is the provision of a medical device that is at least partially formed of a tantalum-tungsten alloy.

Another and/or additional non-limiting object of the present invention is the provision of a medical device that is in the form of a stent.

Still yet another and/or additional non-limiting object of the present invention is the provision of a medical device that includes one or more structural component having varying thicknesses, configurations, and/or surface features so as to affect rate and/or degree at which the medical expands and/or retains its shape in a body passageway.

These and other advantages will become apparent to those skilled in the art upon the reading and following of this description taken together with the accompanying drawings.

### BRIEF DESCRIPTION OF THE DRAWINGS

Reference may now be made to the drawing which illustrates a non-limiting embodiments that the invention may take in physical form and in certain parts and arrangement of parts wherein:

FIG. 1 is a front elevation view of a stent in accordance with the present invention.

FIG. 2 is a front elevation view of another stent in accordance with the present invention.

### DESCRIPTION OF NON-LIMITING EMBODIMENTS

The previously mentioned shortcomings of prior art treatments and medical devices are addressed by the novel medical device and treatment of the disclosure. The treatment in accordance with one present embodiment can include delivering a medical device to a targeted diseased vessel. The medical device can be in the form of a medical device such as, but not limited to, a stent, a graft, a surgical graft (e.g., vascular grafts, etc.), etc.

In one non-limiting aspect, the medical device is particular useful for use in the brain for the treatment of neurological conditions, such as intracranial/neurological stenosis, thrombus or thrombosis, intracranial aneurysms, etc. As used herein, the brain vessels and neurovascular arteries are defined to include any vessel or artery supplying or returning blood from the brain and includes, inter alia, the following arteries: internal carotid artery (ICA); the middle cerebral artery (MCA) including the M1 and M2 segments; the anterior cerebral artery (ACA); and the basilar artery, not discounting all other vessels, etc. The techniques employed to deliver the medical device to a treatment area include, but are not limited to, direct balloon expansion stenting, angioplasty, vascular anastomoses, transplantation, implantation, subcutaneous introduction, minimally invasive surgical procedures, interventional procedures, and any combinations thereof for vascular applications. In one non-limiting embodiment, the medical device is in the form of an intravascular stent. The stent can be an expandable stent that is expandable by an inflatable device inflating against an inside surface of the stent and/or by other means. The stent can have many shapes and forms. Such shapes can include, but are not limited to, stents disclosed in U.S. Pat. Nos. 6,206,916; 6,436,133; and 8,769,794 and all the prior art cited in these patents. Various designs and configurations of stents in such patents are incorporated herein by reference. When the medical device is in the form of a stent, the stent is configured to enable the stent to be inserted into a body passageway in an unexpanded configuration. The stent is configured to expand from an unexpanded configuration to a fully expanded configuration by expansion pressure of over 1 atm. and less than about 6 atm. that is applied against it from the inflatable device. The stent is configured to engage an inner wall of the body passageway to support an opening in the body passageway, for example, by enabling better or proper fluid flow through the vessel. The stent also has sufficient radial strength in the fully expanded position to resist deformation when exposed to an external radial pressure of at least 1.1 atm., and typically 1.1-6 atm. (and all values and ranges therebetween).

Structure

In most cases, stents used to treat coronary conditions and other affected organs are deployed at pressures ranging from 6-15 atm. However, vessels in the brain have much lower pressure deviations than other parts of the body, particularly because intravascular (or “cerebral”) vessels are more complex in their tortuosity and are smaller than other organ vessels. The cerebral vessels cannot handle stents deployed within the standard pressure ranges without added risk for complication and damage to the vessel walls. As such, these prior art stents are not used to treat cerebral vessels. In view of this realization, the present invention is directed to a treatment of neurological conditions using a medical device that is formed of at least 95 wt. % a refractory alloy, such as a tantalum-tungsten alloy material.

The use of the tantalum-tungsten alloy to partially or fully form the medical device enables the medical device to be reduced in thickness and size without the threat of fracturing, as compared to traditional metals and metal alloys used to form stents. A drug and/or polymer coating can optionally be applied to the medical device at even greater thicknesses than previously known, while still not approaching the upper limit of thickness that would cause the medical device to fail when expanded.

Thus, in one non-limiting embodiment, the medical device can be formed of a material that is considered a refractory metal such as a tantalum and tungsten alloy material. Particularly, the medical device is formed at least partially from a non- or low-straining hardening alloy. The medical device is formed from a non-shape memory alloy and a non-self-expanding alloy. By utilizing the intrinsic properties of the refractory alloy material, a medical device such as, but not limited to, a stent can be manufactured in such a way that can at least partially overcome potential problems with stent implantation, such as artery puncture and damage to the lining of the cerebral vessel (causing an artery dissection) in and/or around at the treatment location of the stent.

In another and/or additional non-limiting aspect, the medical device that is at least partially made of a majority of tantalum and tungsten alloy material. Such a medical device has improved physical properties as compared to prior metals and metal alloys used to form stents when such stents are used to treat blood vessel in the brain. The tantalum-tungsten alloy used to at least partially form the medical device (e.g., stent) can expand at substantially lower pressures than the materials commonly used to form prior stents. Therefore, a brain stent or medical device formed at least partially of the disclosed alloy can be deploy at pressure of over 1 atm. and typically about 1.1 to less than 6 atm. In one non-limiting embodiment, the medical device or brain stent is fully expandable from an unexpanded configuration to a fully expanded configuration by an expansion pressure of at least 1.1 atm. In one non-limiting embodiment, the medical device or stent is fully expandable from the unexpanded configuration to the fully expanded configuration by an expansion pressure of no more than 4 atm. and, more particularly, no more than 3 atm. In one non-limiting embodiment, the medical device or brain stent is fully expandable from an unexpanded configuration to a fully expanded configuration by an expansion pressure from about 1.2 atm. to 3 atm., and more particularly from about 1.5 atm. to 2.5 atm.

These one or more improved physical properties of the refractory metal alloy used in the medical device can be achieved in the medical device without having to increase the bulk, volume and/or weight of the medical device, and in some instances can be obtained even when the volume, bulk and/or weight of the medical device is reduced as compared to medical devices that are at least partially formed from aluminum and/or traditional stainless steel or cobalt and chromium alloy materials; however, this is not required.

In still another and/or additional non-limiting embodiment, the medical device comprising the tantalum-tungsten alloy, as compared to traditional materials, can 1) increase the radiopacity of the medical device, 2) limit the range of yield vs. tensile range over current alloys, while providing comparable radial strength, 3) improve the stress-strain properties of the medical device, 4) improve the crimping and/or expansion properties of the medical device, 5) improve the plasticity and deliverability and/or flexibility of the medical device, 6) improve the strength and/or durability of the medical device, 7) increase the hardness of the medical device, 8) improve the longitudinal lengthening properties of the medical device, 9) improve the recoil properties of the medical device (e.g., reduce or eliminate the amount of recoil), 10) reduce the friction coefficient of the medical device, 11) improve the heat sensitivity properties of the medical device, 12) improve the biostability and/or biocompatibility properties of the medical device, 13) enable smaller, thinner and/or lighter weight medical devices to be made, and/or 14) reduce the amount of pressure needed to be applied by the catheter balloon to cause the catheter balloon to fully expand the medical device (e.g., stent). For example, a medical device in accordance with the present invention can be used to treat damaged vessels in hard-to-reach regions (e.g., vessel having less than 7 mm in diameter such as vessels in the brain, etc.), and also to treat complex vessels that curve. The tantalum-tungsten alloy-based material used to form the medical device enables the device to bend at wide angles without cracking. For example, a stent formed from the tantalum-tungsten alloy-based material and having a diameter of no more than 5 mm in the unexpanded position can be configured to bend at acute and obtuse angles ranging from about 0-180°, and typically from 0-150°, and more typically from 0-120°, and even more typically from 0-95°. Such bending range for traditional metals used to form the stent are either not possible or would result in cracking or breaking of the struts, damage the stent during the deployment of the stent, and/or damage to the body passageway during deployment (e.g., lack of the stent to sufficient bend can result in scrapping or puncturing of a curved body passageway during deployment of the stent).

In still another and/or additional non-limiting aspect, the medical device in accordance with the present invention is subject to one or more manufacturing process to impart the desired properties to the medical device. These manufacturing processes can include, but are not limited to, laser cutting, etching, crimping, annealing, drawing, pilgering, electroplating, electro-polishing, chemical polishing, cleaning, pickling, ion beam deposition or implantation, sputter coating, vacuum deposition, etc.

In yet another and/or additional non-limiting aspect, the design characteristics of the medical device are developed into an array of configurations that do not adversely affect the function of such medical device. That is, besides the desired mechanical properties of the medical device (e.g., stent, etc.), the medical device can be configured to interact with the body tissue at the implantation location in a manner such that renewed vessel constrictions do not occur, in particular, vessel constrictions caused by the medical device itself. Re-stenosis (re-constriction of the vessel) should be avoided as much as possible during and after the deployment of the medical device. It is also desirable that the medical device, as far as possible, is responsible for little or no inflammatory effect at the implantation site (e.g., does not scratch, bruise, puncture, etc. the inner surface of the body passageway during deployment and/or expansion of the medical device). In regard to a metal medical device, it is moreover desirable if the composition of the metal alloy used to form the medical device has little or no negative physiological effects on the body passageway. As can be appreciated, the composition of the metal alloy used to form the medical device can have positive physiological effects; however, this is not required.

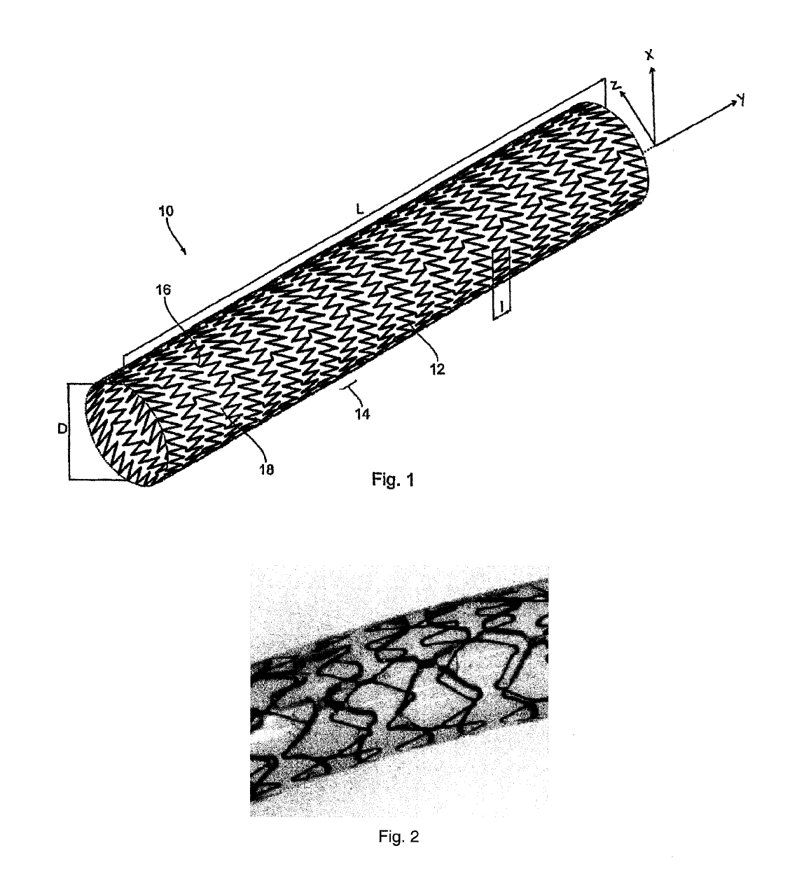

In still yet another and/or additional non-limiting aspect, the configuration of the medical device can take any number of different structures. Thus, with reference to FIG. 1, there is shown an exemplary embodiment medical device in the form of a stent 10. As can be appreciated, the stent can have many other or additional configurations (See FIG. 2).

As illustrated in FIG. 1, the stent 10 and its carrier structure are in the form of a hollow body which is open at its ends and the peripheral wall of which is formed by the carrier structure which in turn is formed by partially folded legs or struts 12. The legs or struts 12 form support portions 14 which are each formed by a leg or strut 12 which is closed in an annular configuration in the longitudinal direction and which is folded in a zig-zag or meander-shaped configuration. The stent is suitable for coronary use such as for blood vessels in the brain, or other types of use.

Stent 10 is formed by a plurality of such carrier structures which occur in succession in the longitudinal direction. The carrier structures are connected together by way of one or more connecting legs 16. As illustrated in FIG. 1, each two connecting legs 16 are mutually adjacent in the peripheral direction and the parts of the carrier structures, which are in mutually opposite relationship between those connecting legs 16, define a mesh 18 of the stent 10. As can be appreciated, the legs or struts of the carrier structure can be oriented in many different configurations. Each mesh 18 encloses a radial opening in the peripheral wall or the carrier structure of the stent 10.

The stent 10 is expandable in the peripheral direction by virtue of the folding of the legs or struts 12 on the carrier structures. The expansion can be achieved for example, by means of a known balloon catheter which at its distal end, has a balloon which is expandable by means of a fluid. The stent 10 can be crimped onto the deflated balloon, in the compressed condition or unexpanded condition. Upon expansion of the balloon, both the balloon and the stent 10 are enlarged or expanded. The balloon can then be deflated again and the stent 10 in its expanded state or condition is released from the balloon. In that way, the catheter can serve simultaneously for introducing the stent 10 into a blood vessel and in particular into a constricted coronary vessel and also for expanding the stent 10 at such desired location.

The geometry of the peripheral wall and legs or struts of the stent will be described by using the co-ordinates shown in FIG. 1, more specifically x as the longitudinal axis of the stent, y as co-ordinates extending radially in the peripheral direction of the stent with respect to the longitudinal direction x, and z as coordinates extending along the width or thickness of the stent.

Many configurations for the support portions and/or connecting legs of the stent lattice are possible. FIG. 2 illustrates a stent having a U, V or Y shaped strut configuration. In various possible non-limiting embodiments, the configurations for the support portions can take multiple forms, including, e.g., the shape of a W, Y, Z, X, U, V and/or S. Various illustrative configurations are disclosed in U.S. Pat. No. 8,769,764 and are incorporated herein by reference. All of these connectors and configurations can have multiple thicknesses along the axis of the medical device and have different angles or degrees of separation. The strut width can vary along the longitudinal length of the strut. The width of the strut is larger at the base or curved portion of the strut. This configuration is utilized to accommodate the different stress points that occur so as not to weaken the device prior to achieving the goal of repairing or supporting a mammalian organ or vessel. The stent of the present invention is suitable for neurological use or other types of use.

In another and/or additional non-limiting aspect of the present invention, the thickness of the legs or struts may vary over the longitudinal length of the leg or strut. The thickness of the tantalum-tungsten alloy material in one portion of the stent can be different from the thickness in another portion of the stent, so as to achieve the desired rate of structural success of the stent in one or more portions of the stent. The varying of the thickness of the support portions and/or connecting legs can be used to controllably expanded or bend the stent in a vessel. The stent can be designed so that the entire stent expands uniformly, or be designed such that one or more portions of the stent expands at differing times and/or rates from one or more other portions of the stent. In one embodiment, the stent 10 is able to go from a small crimped diameter (unexpanded configuration) to a large vessel expansion diameter (expanded configuration). In one aspect of the invention, the stent in the unexpanded configuration is about is about 1 to less than about 5 mm in diameter, and the stent in the stent in said fully expanded state is about 2-12 mm in diameter. In another aspect of the invention, the stent in the unexpanded configuration is about is about 1-4 mm in diameter, and the stent in the stent in said fully expanded state is about 2-12 mm in diameter. In another aspect of the invention, the stent in the unexpanded configuration is about is about 1-3 mm in diameter, and the stent in the stent in said fully expanded state is about 2-10 mm in diameter. In another aspect of the invention, the stent in the unexpanded configuration is about is about 1-2.5 mm in diameter, and the stent in the stent in said fully expanded state is about 2-8 mm in diameter. In another or alternative non-limiting aspect of the invention, the stent in the unexpanded configuration is about 1-1.5 mm in diameter, and the stent in the stent in said fully expanded state is about 2-7 mm in diameter. As can be appreciated, other sizes can be used for stents that are to be deployed in other regions of the body. Specific non-limiting contemplated diameter ranges in accordance with the present invention comprise 1) 1 mm diameter stent in an unexpanded profile or position and expandable to a 3 mm diameter, 2) 1.1 mm diameter stent in an unexpanded profile and expandable to a 3.5 mm diameter, 3) 1.2 mm diameter stent in an unexpanded profile and expandable to a 4 mm diameter, and 4) 1 mm diameter stent in an unexpanded profile and expandable to a 7 mm diameter.

The stent structure can optionally include a section for diverting fluid flow to reduce the risk of intracranial aneurysm.

The stent of the present invention that is at least partially formed of a Ta—W alloy material is designed and configured to be expandable at significantly lower catheter balloon pressures (e.g., 1.1-4 atm.) than compared to prior art stents having similar sizes and thicknesses, but formed of different metal alloys. The stent of the present invention that is at least partially formed of a tantalum-tungsten alloy material is designed and configured to be able to have small unexpanded diameters (e.g., 1-3 mm) and to be expandable without damage to the stent. Such small unexpanded diameters for a stent cannot be successfully formed and subsequently expanded using different metal alloys without damage to the stent during formation and/or expansion.

In yet another and/or additional non-limiting aspect, the exact thickness and/or width variations along the longitudinal axis of the stent will in part depend on the design of the support portions and/or connecting legs of the stent. In addition, the optional use of polymer coatings as well as other layers added to the stent surface can be used to affect one or more properties of the stent. These and other properties and structural design features of the stent are described in U.S. Pat. No. 8,769,794, which are incorporated herein by reference.

In still another and/or additional non-limiting aspect, the medical device such as a stent is partially or fully formed of a refractory alloy (e.g., tantalum-tungsten alloy). In one non-limiting embodiment, the metal portion of the medical device is generally designed to be formed from at least about 25 wt. % of the refractory metal alloy; however, this is not required. In one non-limiting embodiment, the metal portion of the medical device is formed from at least about 40 wt. % of the refractory metal alloy. In another and/or additional non-limiting embodiment, the metal portion of the medical device is formed from at least about 50 wt. % of the refractory metal alloy. In still another and/or additional non-limiting embodiment, the metal portion of the medical device is formed from at least about 60 wt. % of the refractory metal alloy. In yet another and/or additional non-limiting embodiment, the metal portion of the medical includes is formed from at least about 70 wt. % of the refractory metal alloy. In still yet another and/or additional non-limiting embodiment, the metal portion of the medical is formed from at least about 85 wt. % of the refractory metal alloy. In another and/or additional non-limiting embodiment, the metal portion of the medical device is formed from at least about 90 wt. % of the refractory metal alloy. In still another and/or additional non-limiting embodiment, the metal portion of the medical device is formed from at least about 95 wt. % of the refractory metal alloy. In yet another and/or additional non-limiting embodiment, the metal portion of the medical device is formed from at least about 98 wt. % of the refractory metal alloy. In another and/or additional non-limiting embodiment, the metal portion of the medical device is formed from at least about 99 wt. % of the refractory metal alloy. In still another and/or additional non-limiting embodiment, the metal portion of the medical device is formed from at least about 99.5 wt. % of the refractory metal alloy. In yet another and/or additional non-limiting embodiment, the metal portion of the medical device is formed from about 99.8-100 wt. % of the refractory metal alloy.

In yet another and/or additional non-limiting aspect, the refractory metal alloy that is used to form all or a portion of the metal portion of the medical device includes at least about 92.5 wt. % of two refractory metals. In another non-limiting embodiment, the refractory metal alloy that is used to form all or a portion of the metal portion of the medical device includes at least about 95 wt. % of two refractory metals. In another non-limiting embodiment, the refractory metal alloy that is used to form all or a portion of the metal portion of the medical device includes at least about 98 wt. % of two refractory metals. In another non-limiting embodiment, the refractory metal alloy that is used to form all or a portion of the metal portion of the medical device includes at least about 99 wt. % of two refractory metals. In another non-limiting embodiment, the refractory metal alloy that is used to form all or a portion of the metal portion of the medical device includes about 99.5-100 wt. % of two refractory metals.

In another and/or additional non-limiting aspect, a majority weight percent of the refractory alloy is tantalum, and a minority weight percent of tungsten. In one non-limiting embodiment, the metal alloy comprises about 5-10% by weight tungsten and 90-95% tantalum. Specific non-limiting contemplated refractory metal alloys in accordance with the present invention comprise 1) 95% tantalum with 5% tungsten, 2) 92.5% tantalum with 7.5% tungsten, 3) 90% tantalum with 10% tungsten, and 4) 90-97.5% tantalum and 2.5-10% tungsten. In another non-limiting embodiment, at least 99 wt. % of the refractory alloy is formed of tantalum and tungsten. In another non-limiting embodiment, at least 99.5 wt. % of the refractory alloy is formed of tantalum and tungsten. In another non-limiting embodiment, at least 99.9 wt. % of the refractory alloy is formed of tantalum and tungsten. In another non-limiting embodiment, at least 99.95 wt. % of the refractory alloy is formed of tantalum and tungsten. In another non-limiting embodiment, at least 99.99 wt. % of the refractory alloy is formed of tantalum and tungsten.

In still yet another and/or additional non-limiting aspect of the present invention, the medical device that is at least partially formed from the tantalum-tangsten metal alloy can be formed by a variety of manufacturing techniques. U.S. Pat. No. 8,769,764, incorporated herein by reference in its entirety, discloses techniques for manufacturing a medical device according to the disclosure.

The expansion of the stent body member can be accomplished in a variety of manners. Typically, a generally tubular-shaped body member in an unexpanded position is expanded to its second expanded cross-sectional area by a radially, outwardly extending force applied at least partially from the interior region of the body member (e.g., by use of an inflatable device, such as a “balloon”, etc.); however, this is not required. When the second expanded cross-sectional area is variable, the second cross-sectional area is typically dependent upon the amount of radially outward force applied to the body member. The stent can be designed such that the body member expands while retaining the original length of the body member; however, this is not required. The body member can have a first cross-sectional shape that is generally circular so as to form a substantially tubular body member; however, the body member can have other cross-sectional shapes. When the stent includes two of more body members, the two of more body members can be connected together by at least one connector member.

The stent can optionally include rounded, smooth and/or blunt surfaces to minimize and/or prevent damage to a body passageway as the stent is inserted into a body passageway and/or expanded in a body passageway; however, this is not required. The stent can optionally also have its subsurface treated in such a way that it forms gaps below the surface that are sponge-like; however, this is not required. The stent can optionally be treated with gamma, beta and/or e-beam radiation, and/or otherwise sterilized; however, this is not required. The stent can optionally have multiple sections; however, this is not required. The optionally multiple sections of the stent can have a uniform architectural configuration, or can have differing architectural configurations. Each of the sections of the stent can be formed of a single part or formed of multiple parts which have been attached. When a section is formed of multiple parts, typically the section is formed into one continuous piece; however, this is not required.

In still yet another and/or additional non-limiting aspect of the present invention, the medical device optionally can be a drug eluting, drug containing, drug coated, or drug-absorbing stent that can optionally include a drug-coated matrix. While stents commonly used to treat intracranial stenosis and brain aneurysms may not be drug eluting, one or more portions of the medical device can optionally include, contain and/or be coated with one or more biological or bioactive agents that are released into the vessel wall to a) inhibit or prevent thrombosis, in-stent restenosis, vascular narrowing and/or restenosis after the medical device has been positioned in the target vessel; b) at least partially passivate, remove and/or dissolve lipids, fibroblast, fibrin, stem cell, anti-platelet therapy, tPA, limus drugs, taxol etc. causing at least a partial blockage in the target vessel so as to at least partially remove such materials and/or to passivate such vulnerable materials (e.g., vulnerable plaque, etc.) in the target vessel in the region of the medical device and/or downstream of the medical device; and/or c) repair vessels damaged in trauma. As can be appreciated, the one or more optional biological or bioactive agents can have many other or additional uses, such as inhibiting or preventing any adverse biological response by and/or to the medical device that could possibly lead to device failure and/or an adverse reaction by human or animal tissue.

The terms “biological agent” and “bioactive agent” include, but are not limited to, a substance, drug or otherwise formulated and/or designed to prevent, inhibit and/or treat one or more biological problems, and/or to promote the healing in a treated area. Non-limiting examples of biological problems that can be addressed by one or more biological agents include, but are not limited to, neurological conditions and diseases. A non-limiting list of example biological agents is disclosed in U.S. Pat. No. 8,769,794, which is incorporated herein by reference. In still yet another and/or additional non-limiting aspect of the present invention, the medical device optionally can be a drug-emitting stent having a matrix containing an anti-proliferative agent for treating intracranial stenosis. In still yet another and/or additional non-limiting aspect of the present invention, the medical device optionally can be a drug-emitting stent having an intravenous tissue plasminogen activator (tPa) activator and/or derivatives thereof for thrombus. In still yet another and/or additional non-limiting aspect of the present invention, the medical device optionally can be a drug-emitting stent having a gel matrix with a high oxygen content and a normalized saline (e.g., 7.2 pH). In still yet another and/or additional non-limiting aspect of the present invention, the medical device optionally can be a drug-emitting stent containing viable stem cells. In still yet another and/or additional non-limiting aspect of the present invention, the medical device optionally can be a drug-reabsorbing stent having a reabsorbable drug-coated matrix to aid in stem cell release. In one non-limiting example, such a stent optionally can be manufactured to control the degradation rate and/or release rate of omnipotent neuro-stem cells from the stent matrix to mitigate the effects of a stroke.

In another and/or additional non-limiting aspect of the present invention, one or more biological agents optionally can be coated on the medical device by a variety of mechanisms such as, but not limited to, spraying (e.g., atomizing spray techniques, etc.), or can be applied to or affixed to the stent via curing (for example, slightly below body temperature) and crosslinking, etc. to control the release of one or more of the polymer, gels, and/or drugs on the stent matrix.

In another and/or additional non-limiting aspect of the present invention, one or more biological agents on and/or in the medical device, when used on the medical device, can be released in a controlled manner so the area in question to be treated is provided with the desired dosage of biological agent over a sustained period of time. As can be appreciated, controlled release of one or more biological agents on the medical device is not always required and/or desirable. As such, one or more of the biological agents on and/or in the medical device optionally can be uncontrollably released from the medical device during and/or after insertion of the medical device in the treatment area.

It can also be appreciated that one or more biological agents on and/or in the medical device (when used) can be controllably released from the medical device and one or more biological agents on and/or in the medical device can be uncontrollably released from the medical device. As such, the medical device optionally can be designed such that 1) all the biological agent on and/or in the medical device is controllably released, 2) some of the biological agent on and/or in the medical device is controllably released and some of the biological agent on the medical device is non-controllably released, or 3) none of the biological agent on and/or in the medical device is controllably released. The medical device optionally can also be designed such that the rate of release of the one or more biological agents from the medical device is the same or different. The medical device optionally can also be designed such that the rate of release of the one or more biological agents from one or more regions on the medical device is the same or different.

In still another and/or additional non-limiting aspect of the present invention, non-limiting arrangements that can be used to control the release of one or more biological agents from the medical device, when such controlled release is desired, include a) at least partially coating one or more biological agents with one or more polymers, b) at least partially incorporating and/or at least partially encapsulating one or more biological agents into and/or with one or more polymers, and/or c) inserting one or more biological agents in pores, passageway, cavities, etc. in the medical device and at least partially coating or covering such pores, passageway, cavities, etc. with one or more polymers. As can be appreciated, other or additional arrangements can be used to control the release of one or more biological agent from the medical device.

In yet another and/or additional non-limiting aspect of the present invention, one or more polymers optionally can be used to at least partially control the release of one or more biological agents from the medical device. The one or more polymers (when used) can be porous or non-porous. As such, the one or more biological agents on the medical device can 1) be coated on one or more surface regions of the medical device, and/or 2) form at least a portion or be included in at least a portion of the structure of the medical device. When the one or more biological agents are coated on the medical device, the one or more biological agents can 1) be directly coated on one or more surfaces of the medical device, 2) be mixed with one or more coating polymers or other coating materials and then at least partially coated on one or more surfaces of the medical device, 3) be at least partially coated on the surface of another coating material that has been at least partially coated on the medical device, and/or 4) be at least partially encapsulated between a) a surface or region of the medical device and one or more other coating materials and/or b) two or more other coating materials.

In still yet another and/or additional non-limiting aspect of the present invention, one or more portions of a support portion (e.g., leg or strut) and/or a connecting leg of the stent optionally can include one or more passageways. These one or more passageways can be used to alter one or more physical properties of the support portion and/or a connecting leg (e.g., strength, bendability, etc.) and/or be used to contain one or more polymers and/or biological agents. The internal passageways optionally can be coated with a polymer along with the surface of the medical device or the interior of the passageway can also or alternately be filled with a biological agent. These passageways can be filled in various ways. One non-limiting method is to introduce the biological agent or polymer onto the medical device is in a vacuum chamber. The reduced pressure will draw the biological agent or polymer into the internal passageways. As can be appreciated, other methods can be used to incorporate a polymer and/or biological agent in the cavity or internal passageway. The biological agent or polymer may or may not be applied at the time and point of use, such as at the time the medical device is positioned in the target vessel, in a clean lab, or may be introduced into the stent matrix at the time of manufacture.

In another and/or additional non-limiting aspect of the present invention, many coating arrangements optionally can be used on the medical device.

The medical device, when including and/or is coated with one or more biological agents, can include and/or can be coated with one or more biological agents that are the same or different in different regions of the medical device and/or have differing amounts and/or concentrations in differing regions of the medical device. For example, the medical device can a) be coated with and/or include one or more biological agents on at least one portion of the medical device and at least another portion of the medical device is not coated with and/or includes biological agents; b) be coated with and/or include one or more biological agents on at least one portion of the medical device that is different from one or more biological agents on at least another portion of the medical device; c) be coated with and/or include one or more biological agents at a concentration on at least one portion of the medical device that is different from the concentration of one or more biological agents on at least another portion of the medical device; etc.

In still another and/or additional non-limiting aspect of the present invention, one or more surfaces of the medical device optionally can be treated to achieve the desired coating properties of the one or more biological agents and one or more polymers coated on the medical device. As can be appreciated, one or more surfaces of the medical device optionally can be treated to achieve the desired surface properties of the medical device when the medical device is not a coated device. Such surface treatment techniques include, but are not limited to, cleaning, buffing, smoothing, etching (chemical etching, plasma etching, etc.), etc. When an etching process is optionally used, various gasses can be used for such a surface treatment process such as, but not limited to, carbon dioxide, nitrogen, oxygen, Freon, helium, hydrogen, etc. The plasma etching process can be used to clean the surface of the medical device, change the surface properties of the medical device so as to affect the adhesion properties, lubricity properties, etc. of the surface of the medical device. As can be appreciated, other or additional surface treatment processes optionally can be used.

In one non-limiting manufacturing process, one or more portions of the medical device are optionally cleaned and/or plasma etched; however, this is not required. Plasma etching optionally can be used to clean the surface of the medical device, and/or to form one or more non-smooth surfaces on the medical device to facilitate in the adhesion of one or more coatings of biological agents and/or one or more coatings of polymer on the medical device.

In yet another and/or additional non-limiting aspect of the invention, the medical device optionally can include a marker material that facilitates enabling the medical device to be properly positioned in a body passageway. The marker material is typically designed to be visible to electromagnetic waves (e.g., x-rays, microwaves, visible light, infrared waves, ultraviolet waves, etc.); sound waves (e.g., ultrasound waves, etc.); magnetic waves (e.g., MRI, etc.); and/or other types of electromagnetic waves (e.g., microwaves, visible light, infrared waves, ultraviolet waves, etc.). In one non-limiting embodiment, the marker material is visible to x-rays (i.e., radiopaque). The marker material (when used) can form all or a portion of the medical device.

In another and/or additional non-limiting aspect of the present invention, other or additional manufacturing techniques can be used. The medical device optionally can include one or more surface structures (e.g., pore, channel, pit, rib, slot, notch, bump, teeth, well, hole, groove, etc.). These structures can be at least partially formed by other types of technology.

Although the medical device of the present invention can be designed and configured to reduce or eliminate the need for long periods of body-wide therapy after the medical device has been inserted in the treatment area, the optional use of one or more biological agents can be used in conjunction with the medical device to enhance the success of the medical device and/or reduce or prevent the occurrence of in-stent restenosis, vascular narrowing, and/or thrombosis.

In one non-limiting embodiment of the invention, the stent can be formed by use of several processes. For instance, a tube of tantalum-tungsten alloy can be formed by a vacuum arc melting process in which the formed alloy is extruded and processed into a rod, or metal powder can be consolidated into the alloy isostatic pressing and sintering at high temperatures under a vacuum. The formed ingot can be cut into lengths of about 20-48 inches (i.e., 36 inches). The diameter of the ingot may be up to about 1 in. in diameter (e.g., 0.0625 inches). The solid rod can be drilled to form a tube having the desired inner and outer diameters and wall thickness. The stent can be formed by laser cutting or other cutting processes. Other processing steps for the tantalum-tungsten alloy that can be used in the present invention are disclosed in U.S. Pat. Publ. No. 2006/0264914, which is incorporated herein.

In one non-limiting embodiment of the present invention, a blood vessel in the brain is repaired with a medical device that is at least partially formed of a refractory alloy and is positioned in the target vessel. The diameter of blood vessels in the brain that are to be treated by the medical device of the present invention have a diameter of no greater than 12 mm, and typically no greater than 10 mm, and more typically no greater than 8 mm. As such, the medical device of the present invention is configured to be used in much smaller blood vessels than are typically treated.

Treatment of Intracranial Artery Stenosis

Intracranial artery stenosis is a narrowing of an artery in the brain that can lead to stroke. Stenosis is caused by a buildup of plaque inside the artery wall that reduces blood flow to the brain, which can lead to severe symptoms and a high risk of stroke, brain damage, and death. Intracranial artery stenosis can result in an ischemic stroke if the plaque narrows (or occludes or blocks) the artery and reduces the blood flow to the brain; if the plaque deforms the artery wall, blood clots can form and block the blood flow to the brain; or if the plaque ruptures and breaks away and lodges in a smaller artery and blocks blood flow to the brain.

According to one non-limiting embodiment of the invention, the intracranial artery stenosis is treated using an intravascular stent in accordance with the present invention comprising a strut pattern (e.g. laser-cut from a non-clad metal tube body) that is at least partially formed of a refractory alloy that itself is formed of at least 95 wt. % of two refractory metals of tantalum and tungsten. In one embodiment, such alloy is formed of majority weight percent of tantalum (over 50 wt. %) and a minority of tungsten (less than 50 wt. %). The stent in the unexpanded configuration is about 1 mm to less than about 5 mm in diameter, and about 2-12 mm in diameter in the fully expanded state, and has a recoil after being fully expanded of no more than 5%.

One property of a stent formed of the refractory alloy in accordance with the present invention is that it is expandable by an inflatable device (e.g., catheter balloon, etc.) at an ultra-low pressure that is less than 6 atm. and, more particularly, over 1 atm. and up to 5 atm., and still more particularly from 1.1 atm. to 4 atm., and even more particularly from 1.1 atm. to 3 atm. Such expansion pressures are significantly lower than those used to expand prior art stents that require balloon expansion. Prior art metals and alloys that are used in non-self-expanding and non-memory alloy stents typically require over 6 atm. to expand the stent.

In one embodiment of the invention, a stent formed is a non-memory alloy and a non-self-expandable refractory alloy (e.g., tantalum-tungsten alloy) and is expandable in the peripheral direction by virtue of the folding of the support portions (e.g., legs or struts). The stent can optionally be is crimped, in the compressed condition or unexpanded position, onto a deflated balloon catheter. Generally, the metal tube body of the stent is configured to enable the stent to be inserted into a body passageway in the unexpanded position. In one non-limiting configuration, the crimp profile of the stent is less than 5 mm in diameter, and typically approximately 1-2 mm in diameter. The configuration of the refractory alloy (e.g., tantalum-tungsten alloy) enables the stent to expand to a deployed profile of about up to 12 mm, and typically about 3-7 mm to cause the outer surface of the stent to engage an inner wall of the body passageway in the brain. The amount of recoil of the stent after being expanded is no more than 5%, and typically less than 4%, and more typically less than 3%. The strut thickness of the stent is generally no more than about 0.0029 inches, and typically about 0.0012-0.0022 inches, and more typically about 0.0012-0.002 inches; and the strut width is generally about 0.0002-0.0029 inches, and typically about 0.0004-0.002 inches. Generally, the ratio of the thickness of the strut to the width of the strut is 4.5:1 to 1.01:1, and typically about 4:1 to 1.01:1.

When the unexpanded stent is placed in position in the damaged vessel in a brain, a fluid is applied to the catheter balloon at a pressure of less than 6 atm., typically about 1.1 atm. to 4 atm., to cause the stent to expand from the unexpanded position to the expanded position or fully expanded position. Upon expansion of the balloon catheter, both the balloon catheter and the stent are enlarged and expanded. The stent is configured that when expanded, the outer surface of the stent engages the inner wall of the brain blood vessel to secure the stent in position in the brain blood vessel. The stent in the fully expanded positioned is no more than 5% greater than the inner diameter of the brain blood vessel so as to reduce the forces applied to the blood vessel and to reduce the chances of damage or rupture of the brain blood vessel. The stent is also configured that once the balloon catheter is deflated and removed from the stent, the recoil of the stent is less than 5% so that the stent, after any recoil, still has the outer surface engaging the inner surface of the brain blood vessel so as to maintain the stent in a secure position in the brain blood vessel.

After the stent has been expanded, the balloon catheter can be deflated and the brain stent released from the balloon catheter. As such, the balloon catheter can serve simultaneously for introducing the stent into a blood vessel and in particular into a constricted cerebral vessel and also for expanding the stent at that target location.

The stent of the present invention is configured such that the stent in the expanded position has sufficient radial strength to resist deformation and to maintain the brain blood vessel in an open position when exposed to an external radial pressure of at least 1.1 atm. and typically less than about 6 atm.

Treatment of intracranial artery stenosis can be accomplished by virtue of repairing the vessel wall. The deployment of refractory alloy (e.g., Ta—W alloy) stent under the ultra-low pressure (e.g., less than 6 atm.) also reduces the risk of vessel wall injury, which also decreases the risk of inflammatory response and other conditions during recovery.

In still another and/or additional non-limiting aspect of the invention, the treatment optionally can be used in conjunction with one or more other biological agents that are not on the stent. For instance, the success of the treatment using the refractory alloy (e.g., Ta—W alloy) stent can be improved by infusing, injecting or orally consuming one or more biological agents. Such biological agents can be the same and/or different from the one or more biological agents optionally on and/or in the medical device. Such use of one or more biological agents are commonly used in systemic treatment of a patient after a medical procedure, such as body-wide therapy, after the medical device has been inserted in the treatment area can be reduced or eliminated by use of the novel alloy.

Treatment of Intracranial Aneurysms

In another non-limiting embodiment of the present invention, a blood vessel in the brain is repaired when a medical device that is at least partially formed of a refractory alloy is positioned in the target vessel for treatment of intracranial aneurysms.

Intracranial or cerebral aneurysm is a bulging or ballooning in a weak area of the vessel wall that supplies blood to the brain. Should the aneurysm rupture, blood is released into the skull, causing a hemorrhagic stroke. When the aneurysm ruptures, a life-threatening hemorrhage can result and cause brain damage or death if it is not treated immediately. Treatment of an unruptured intracranial aneurysm may prevent the rupture.

According to one non-limiting embodiment of the invention, the intracranial aneurysm is treated using an intravascular stent having the same or similar properties as the stent described above with regard to the treatment of intracranial artery stenosis.

The intracranial aneurysm can be treated by delivery of a brain stent to the damaged portion of the vessel affected by the unruptured brain aneurysm by application of ultra-low pressure. The stent can be expanded and deployed under substantially lower pressure than the materials commonly used to form prior art stents.

The positioning and delivery of the stent under low pressure of enables the mesh portion of the stent to cover the neck of the aneurysm. In one embodiment, the stent optionally can include a partial or complete elastomeric cover extending along a distal to proximal end of the stent. In one embodiment, the stent can also optionally include an aneurysm flow disrupter, which is released into the aneurysm to block it from circulation and cause the blood to clot, which aims to destroy the aneurysm. In one non-limiting embodiment, a plurality of fibers incorporated on the stent can block the aneurysm flow by forming a partial blood flow diversion. The stent can be positioned to stabilize the vessel and the flow disrupter prevents the aneurysm from bulging or ballooning back into the vessel. Treatment of intracranial aneurysms using the disclosed approach of the embodiment transforms the target vessel by virtue of repairing the vessel wall. The deployment of the stent under the ultra-low pressure reduces the risk of vessel wall injury, which in turn decreases the risk of inflammatory response and other conditions during recovery.

In still another and/or additional non-limiting aspect of the invention, the treatment optionally can be used in conjunction with one or more other biological agents that are not on the medical device. For instance, the success of the treatment using the stent can be improved by infusing, injecting or consuming orally one or more biological agents. Such biological agents optionally can be the same and/or different from the one or more biological agents on and/or in the medical device. Such use of one or more biological agents are commonly used in systemic treatment of a patient after a medical procedure, such as body-wide therapy, after the medical device has been inserted in the treatment area can be reduced or eliminated by use of the novel alloy.

Treatment of Thrombus

In another non-limiting embodiment of the present invention, a blood vessel in the brain is repaired when a medical device that is at least partially formed of a refractory alloy is positioned in the target vessel for treatment of thrombus.

Thrombus is a blood clot (ischemic stroke) in the brain's arteries, which prevents blood from flowing through the arteries and capillaries of the brain. The resulting lack of blood flow deprives the affected brain tissue of nutrition and oxygen. Ischemic stroke comprises about eighty-three percent (83%) of all strokes.

According to one embodiment of the invention, the thrombus can be treated using an intravascular stent having the same or similar properties as the stent described above with regard to the treatment of intracranial artery stenosis.

The thrombus can be treated by delivery of the brain stent to the damaged portion of the vessel affected by the clot by application of ultra-low pressure. Such a stent enables the stent to expand and deploy under substantially lower pressure than the materials commonly used to form prior stents.

Treatment of thrombus using the disclosed approach of the embodiment transforms the target vessel by virtue of clearing the vessel. The deployment of the stent under the low pressure also reduces the risk of vessel wall injury, which also decreases the risk of inflammatory response and other conditions during recovery.

In still another and/or additional non-limiting aspect of the invention, the treatment optionally can be used in conjunction with one or more other biological agents, such as thrombolytic drug therapy (tPA). For instance, the success of the treatment using the stent can be improved by performing the stenting in conjunction with one or more biological agents. For instance, the success of the treatment using the stent can be further improved by performing the stenting within 12 hours and, more preferably 8, hours after the onset of symptoms.

The exemplary embodiment has been described with reference to the preferred embodiments. Obviously, modifications and alterations will occur to others upon reading and understanding the preceding detailed description. It is intended that the exemplary embodiment be construed as including all such modifications and alterations insofar as they come within the scope of the appended claims or the equivalents thereof.

1. An intravascular stent for use in blood vessel of the brain that having an inner diameter of no more than 12 mm, said stent comprising a strut pattern having a plurality of struts formed in a non-clad metal body, at least a portion of said metal body is formed of a refractory alloy, at least 95 weight percent of said refractory alloy is formed of two refractory metals, said refractory alloy is a non-shape memory alloy and a non-self-expanding alloy, said stent having a wall thickness, strut thickness and strut configuration to enable said stent to be expanded from an unexpanded configuration to a fully expanded configuration by expansion pressure of at least 1 atm. and less than about 6 atm. by use of an inflatable device inflating against an inside surface of said metal body, said metal body in said unexpanded configuration having a diameter of no more than 5 mm to enable said stent to be inserted into a blood vessel, said stent in said fully expanded positioned having a diameter of no more than 12 mm to enable an outer surface of said stent to engage an inner wall of the blood vessel to thereby support an opening in the blood vessel, said stent having a recoil of no more than 5% after being expanded in the blood vessel, said stent having sufficient radial strength in said fully expanded position to resist deformation when exposed to an external radial pressure greater than 1 atm.

2. The stent as defined in claim 1, wherein 98-100 wt. % of said refractory alloy is formed of two refractory metals.

3. The stent as defined in claim 1, wherein 99-100 wt. % of said refractory alloy is formed of two refractory metals.

4. The stent as defined in claim 1, wherein said two refractory metals in said refractory alloy are tantalum and tungsten.

5. The stent as defined in claim 2, wherein said two refractory metals in said refractory alloy are tantalum and tungsten.

6. The stent as defined in claim 4, wherein said refractory alloy consists essentially of 90-97.5 wt. % tantalum and a balance weight percent of tungsten.

7. The stent as defined in claim 5, wherein said refractory alloy consists essentially of 90-97.5 wt. % tantalum and a balance weight percent of tungsten.

8. The stent as defined in claim 1, wherein said stent is expandable from an unexpanded configuration to a fully expanded configuration by said expansion pressure of at least 1.1 atm. and up to 5 atm.

9. The stent as defined in claim 2, wherein said stent is expandable from an unexpanded configuration to a fully expanded configuration by said expansion pressure of at least 1.1 atm. and up to 5 atm.

10. The stent as defined in claim 1, wherein said stent is expandable from an unexpanded configuration to a fully expanded configuration by said expansion pressure of at least 1.1 atm. and up to 3 atm.

11. The stent as defined in claim 2, wherein said stent is expandable from an unexpanded configuration to a fully expanded configuration by said expansion pressure of at least 1.1 atm. and up to 3 atm.

12. The stent as defined in claim 1, wherein said stent is expandable from an unexpanded configuration to a fully expanded configuration by said expansion pressure of at least 1.1 atm. and up to 2 atm.

13. The stent as defined in claim 2, wherein said stent is expandable from an unexpanded configuration to a fully expanded configuration by said expansion pressure of at least 1.1 atm. and up to 2 atm.

14. The stent as defined in claim 1, wherein said stent in said unexpanded configuration is about 1-4 mm in diameter, and said stent in the stent in said fully expanded state is about 2-12 mm in diameter.

15. The stent as defined in claim 2, wherein said stent in said unexpanded configuration is about 1-4 mm in diameter, and said stent in the stent in said fully expanded state is about 2-12 mm in diameter.

16. The stent as defined in claim 1, wherein said stent in said unexpanded configuration is about 1-3 mm in diameter, and said stent in the stent in said fully expanded state is about 2-10 mm in diameter.

17. The stent as defined in claim 2, wherein said stent in said unexpanded configuration is about 1-3 mm in diameter, and said stent in the stent in said fully expanded state is about 2-10 mm in diameter.

18. The stent as defined in claim 1, wherein said stent in said unexpanded configuration is about 1-2.5 mm in diameter, and said stent in the stent in said fully expanded state is about 2-8 mm in diameter.

19. The stent as defined in claim 2, wherein said stent in said unexpanded configuration is about 1-2.5 mm in diameter, and said stent in the stent in said fully expanded state is about 2-8 mm in diameter.

20. The stent as defined in claim 1, wherein said stent in said unexpanded configuration is about 1-1.5 mm in diameter, said stent in said stent in said fully expanded state is about 2-7 mm in diameter.

21. The stent as defined in claim 2, wherein said stent in said unexpanded configuration is about 1-1.5 mm in diameter, said stent in said stent in said fully expanded state is about 2-7 mm in diameter.

22. The stent as defined in claim 1, wherein said recoil is no more than 4%.

23. The stent as defined in claim 2, wherein said recoil is no more than 4%.

24. The stent as defined in claim 1, wherein said recoil is no more than 3%.

25. The stent as defined in claim 2, wherein said recoil is no more than 3%.

26. The stent as defined in claim 1, wherein said recoil is no more than 2%.

27. The stent as defined in claim 2, wherein said recoil is no more than 2%.

28. The stent as defined in claim 1, wherein each of said struts has a strut thickness of about 0.001-0.0029 inches, and a strut width of 0.0002-0.0029 inches.

29. The stent as defined in claim 2, wherein each of said struts has a strut thickness of about 0.001-0.0029 inches, and a strut width of 0.0002-0.0029 inches.

30. The stent as defined in claim 1, wherein a ratio of a thickness of said strut to a width of said strut is 5:1 to 1:1.

31. The stent as defined in claim 2, wherein a ratio of a thickness of said strut to a width of said strut is 5:1 to 1:1.

32. The stent as defined in claim 1, wherein said metal body includes one or more bioactive agents.

33. The stent as defined in claim 2, wherein said metal body includes one or more bioactive agents.

34. The stent as defined in claim 31, wherein an exterior surface of said metal body includes surface modifications operative to receive said one or more bioactive agents.

35. The stent as defined in claim 33, wherein an exterior surface of said metal body includes surface modifications operative to receive said one or more bioactive agents.

36. A method for treating a disease in the blood vessel of the brain using said as defined in claim 1, the method comprising:
positioning said stent in the diseased blood vessel in the brain while said stent is in said unexpanded configuration;
expanding said stent at a low pressure of less than about 6 atm. from said unexpanded configuration to said fully expanded configuration using an inflatable device positioned in an interior of said stent, said stent in said fully expanded position having an outer surface of said stent engaging an inner surface of said blood vessel and thereby causing said stent to be secured in a static positioned in said blood vessel; and
deflating said inflatable device and removing said deflated inflatable device from said stent;
wherein said stent is configured to have sufficient radial strength in said fully expanded position to resist deformation when exposed to an external radial pressure of at least 1.1 atm. by said blood vessel, and wherein said disease in said blood vessel is a neurovascular disease that consists of intracranial artery stenosis, intracranial aneurysm, thrombus, or a combination of the above.

37. A method for treating a disease in the blood vessel of the brain using said as defined in claim 2, the method comprising:
positioning said stent in the diseased blood vessel in the brain while said stent is in said unexpanded configuration;
expanding said stent at a low pressure of less than about 6 atm from said unexpanded configuration to said fully expanded configuration using an inflatable device positioned in an interior of said stent, said stent in said fully expanded position having an outer surface of said stent engaging an inner surface of said blood vessel and thereby causing said stent to be secured in a static positioned in said blood vessel; and
deflating said inflatable device and removing said deflated inflatable device from said stent;
wherein said stent is configured to have sufficient radial strength in said fully expanded position to resist deformation when exposed to an external radial pressure of at least 1.1 atm. by said blood vessel, and wherein said disease in said blood vessel is a neurovascular disease that consists of intracranial artery stenosis, intracranial aneurysm, thrombus, or a combination of the above.

38. The method as defined in claim 36, wherein said inflatable device is a balloon or inflatable catheter.

39. The method as defined in claim 37, wherein said inflatable device is a balloon or inflatable catheter.

40. The method as defined in claim 36, wherein said step of expanding occurs at a pressure of at least 1.1 atm. and up to 4 atm.

41. The method as defined in claim 37, wherein said step of expanding occurs at a pressure of at least 1.1 atm. and up to 4 atm.

42. The method as defined in claim 36, wherein said step of expanding occurs at a pressure of at least 1.1 atm. and up to 3 atm.

43. The method as defined in claim 37, wherein said step of expanding occurs at a pressure of at least 1.1 atm. and up to 3 atm.

44. The method as defined in claim 36, wherein said step of expanding occurs at a pressure of at least 1.1 atm. and up to 2 atm.

45. The method as defined in claim 37, wherein said step of expanding occurs at a pressure of at least 1.1 atm. and up to 2 atm.
